(19) United States
(12) Patent Application Publication
(10) Pub. No.: US 2026/0345083 A1
(43) Pub. Date: Nov. 5, 2026

(54) SYSTEMS AND METHODS FOR REDUCING TRIBOELECTRIC NOISE

(71) Applicant: COREMAP, INC., Burlington, MA (US)

(72) Inventor: Peter S. Spector, Colchester, VT (US)

(21) Appl. No.: 19/357,948

(22) Filed: Oct. 14, 2025

Related U.S. Application Data
(60) Provisional application No. 63/797,542, filed on Apr. 30, 2025.

(51) Int. Cl. A61B 5/00; A61B 5/287; A61B 5/346

(52) U.S. Cl. CPC A61B 5/7217; A61B 5/287; A61B 5/346; A61B 5/6852; A61B 5/6869; A61B 2560/0468

(57) ABSTRACT

The invention provides systems and methods for removing triboelectric noise from an electrical signal. The invention relies on the premise that triboelectric noise is present in similarly situated wires such that noise in one set of wires is indicative of noise in another set of wires. The systems comprise a detector connected to a set of noise wires for identifying one or more intervals of noise above a threshold level, and a subprocessor connected to the detector and a set of signal wires for removing the one or more intervals from signals from the signal wires to provide a cleaned signal.

### TECHNICAL FIELD

The disclosure relates to systems and methods for reducing triboelectric noise from catheter signals. Some embodiments of the disclosure provide applications for use in cardiac catheter signal processing.

### BACKGROUND

The triboelectric effect describes electric charge transfer between two objects when they contact or slide against each other. It can occur with different materials, such as the sole of a shoe on a carpet, or between two pieces of the same material. It is ubiquitous effect that occurs with differing amounts of charge transfer (also referred to as “tribocharge”) for all solid materials. In addition, there is evidence that tribocharging can occur between combinations of solids, liquids and gases, for instance liquid flowing in a solid tube or an aircraft flying through air.

In medical cable assemblies, triboelectric noise is generated when the conductors, insulation, and fillers rub against each other as the cables are flexed during movement. Keeping triboelectric noise at acceptable levels is traditionally resolved by careful material selection, cable design, and manufacturing processes. But these methods are still incapable of eliminating this natural phenomenon.

Unfortunately, when measuring low-level signals, any noise in a cable or wire may be problematic as the noise can cause difficulty in interpreting electrical signals carried by a cable or wire as separation of the signal and noise is impossible. For example, intracardiac catheters that collect electrical signals suffer from triboelectric noise when the wires in the catheter shaft rub against each other as the shaft is deformed during positioning and operation. Similarly, noise in an ECG or other medical signal may make accurate diagnosis difficult or even impossible. Accordingly, there is a need for methods and systems to remove triboelectric noise from electrical signals.

### SUMMARY OF THE INVENTION

The invention provides methods and systems for removing triboelectric noise from electrical signals. Methods and systems of the invention make use of a set of noise wires, in addition to signal wires, in order to reduce or eliminate triboelectric noise. While the signal wires carry the electrical signal of interest, the noise wires are positioned close to the signal wires such that any triboelectric noise generated in the signal wires is also generated in the noise wires.

Methods of the disclosure measure a noise signal through the set of noise wires and a raw signal through the set of signal wires. The raw signal includes the electrical signal of interest and also includes any noise signal. The noise signal is processed to identify one or more noise intervals when the noise signal exceeds a threshold level. These noise intervals are removed from the raw signal to provide a cleaned signal. In some embodiments, the cleaned signal is further analyzed. Thus, methods of the invention function as a gate to reduce/remove noise.

Systems and methods of the disclosure are applicable to any electrical signal carried by wires that may suffer from triboelectric noise. In some embodiments, the signal is a cardiac activation signal. In some embodiments, the signal is collected by a catheter. In those embodiments, the set of noise wires and the set of signal wires preferably run the length of the catheter. Accordingly, any triboelectric noise created by deforming or moving the catheter during positioning is able to be removed from the signal provided through the signal wires.

Systems of the disclosure comprise a detector and a processor connected to the set of signal wires and the set of noise wires. The detector is connected to the set of noise wires and configured to identify one or more intervals of noise above a threshold level based on signals from the noise wires. The processor is connected to the detector and the set of signal wires and configured to remove the intervals of noise from the signal wires to provide a cleaned signal. In some embodiments, the system further comprises a catheter body containing the set of noise wires and the set of signal wires.

In some embodiments, systems further comprise additional signal processing elements for processing or analyzing the cleaned signal. The signal processing elements may include a computer system comprising at least one microprocessor coupled to a memory subsystem and a display device. The computer system may be operable to use the cleaned signal to provide an output through the display device. For example, the signal processing elements may provide, on the display device, a model of a heart in which an electrode array attached to the signal wires takes measurements. In some embodiments, the signal processing elements may include one or more of a multiplexer, an amplifier, or an analog-to-digital converter.

Reference is made throughout the disclosure to embodiments which relate to cardiac catheters and cardiac mapping. While exemplary of some embodiments, those skilled in the art will recognize that the methods and systems disclosed herein may be applicable to removing triboelectric noise from any signal which suffers therefrom.

### BRIEF DESCRIPTION OF DRAWINGS

FIG. 1 shows a system for monitoring cardiac rhythm.

FIG. 2 shows steps of a method of analyzing cardiac rhythm.

FIG. 3 shows obtaining the measurements of the electrical activity of a heart.

### DETAILED DESCRIPTION

The invention relates to methods and systems useful to remove triboelectric noise from an electrical signal. Systems and methods of the invention use a set of signal wires to transmit the electrical signal and a set of noise wires to collect and transmit triboelectric noise. The triboelectric noise transmitted by the set of noise wires is also present in the electrical signal transmitted by the set of signal wires. Systems and methods of the invention detect and/or identify one or more intervals of noise above a threshold level based on signals from the noise wires. Systems and methods of the invention then remove the one or more intervals of noise from the electrical signal of the set of signal wires to provide a cleaned signal. Systems and methods of the invention may further analyze the cleaned signal. Threshold levels are determined empirically or at the discretion of the user.

Among other things, the invention makes use of the insight that similarly-situated wires experience triboelectric noise at the same time. A set of noise wires, even if unconnected to any other electrical elements, will experience similar triboelectric noise as a set of signal wires that are manipulated in the same way. Based on that insight, the inventors have identified that a signal passed along a set of signal wires which are also run alongside a set of noise wires can be cleaned to remove triboelectric noise from the catheterization signal, thereby improving signal fidelity and accuracy of measurements/analysis based on the cleaned signal.

Systems and methods of the invention have particular utility in mapping cardiac activity including, for example, when generating a viewable map with a representation of a heart with a graphic depiction of cardiac activity in the heart. Using the invention, the maps generated are more accurate and reproduceable as the cleaned signal does not include triboelectric noise (generally caused by external factors from the heart). In a preferred embodiment, signal wires and noise wires run concomitantly along a cardiac catheter comprising a terminal electrod array, and a processor in communication with the catheter removes noise by subtracting signal received by the noise wires.

In some embodiments, these maps may be used to guide additional mapping and/or treatment. For example, during a cardiac mapping procedure, those maps may be displayed (e.g., on a monitor) allowing a clinician to decide in what areas of the heart to gather additional data. Similar, during an ablation procedure, because the potentially independent incidences of atrial fibrillation are drawn or shown on independent maps, a clinician may be able to identify a focal or driver or re-entry circuit associated with each incidence or regime of atrial fibrillation, providing the clinician with the necessary information about location to ablate to treat the condition.

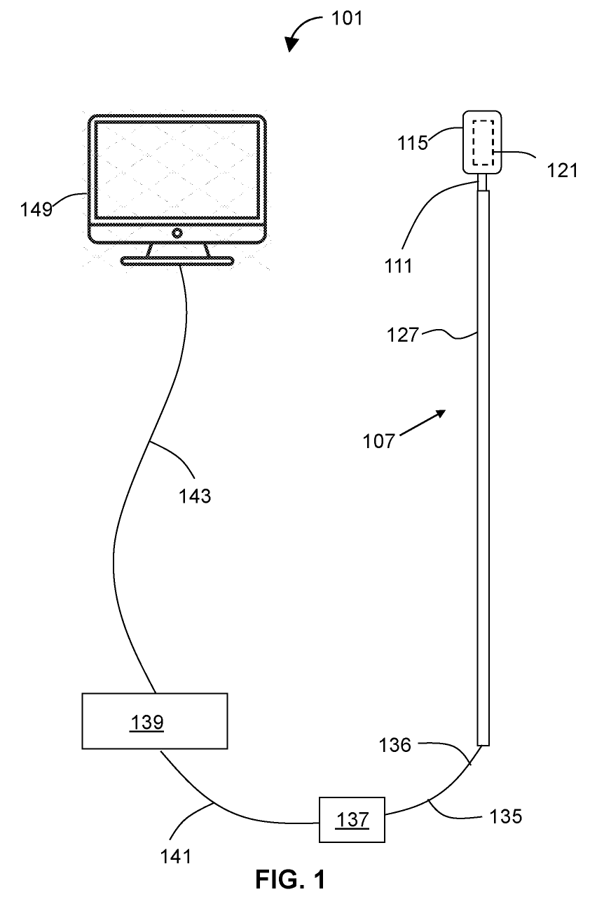

FIG. 1 shows a macrosystem 101 for monitoring cardiac rhythms. The cardiac rhythms may be indicative of arrythmias or fibrillations. The macrosystem 101 includes a catheter 107 that may generally include a shaft 111 extending to a head 115 carrying an electrode array 121. Electrodes of the electrode array 121 may optionally be connected via leads or at least one signal wire 135 to a system 137 of the invention. At least one noise wire 136 is also present in the shaft 111 and connected to the system 137. The shaft 111 may extend through, and out of a distal end of, an introducer sheath 127.

The system 137 may have a data connection 141 to a processor 139, which in turn may have a data connection 143 to a computer system 149. The computer system 149 may record cleaned signals from the system 137 and/or the processor 139 and then analyze the signals to identify locations and incidences of arrythmias and/or fibrillations in the heart. The computer system 149 has at least one computer processor coupled to a memory subsystem having instructions therein executable by the computer processor to cause the computer system 149 to analyze and report information based on the cleaned signal.

The system 137 performs the functions of the methods of the disclosure described herein. The system 137 comprises a detector connected to a set of noise wires. The detector is configured to identify one or more intervals of noise above a threshold level based on signals from the noise wires. The system 137 further comprises a subprocessor connected to the detector and a set of signal wires. The subprocessor is configured to remove the one or more intervals from signals from the signal wires to provide a cleaned signal.

As discussed above, the system 137 may be connected to a processor 139. While FIG. 1 illustrates the processor as being positioned “downstream” of the system 137, it is equally envisioned to position the processor 139 “upstream” of the system, or for the functions of the processor 139 to be performed both upsteam and downstream of the system 137 by more than one element.

The processor 139 is preferably a special-purpose device built specifically to perform signal processing on the cleaned signal. The processor 139 may be a hardware unit standing bed-side in a catheterization laboratory ("cath lab") or hospital or clinical facility. The processor 139 may mediate signals between the electrode array 121 and a computer system 149 (which commonly in a cath lab may not be in the same room and may use cloud or server computers that are remote (other city or state) connected thereto to a control computer terminal in an observation room of the cath lab).

The processor 139 may receive a set of noise wires, a set of signal wires, or the a data connection 141 from the system 137. The processor 139 may include one or more of a multiplexer, an amplifier, and an analog-to-digital (ADC) converter (in any order). The processor 139 may further include a data connection 143 (e.g., one or more wires) to transmit the converted signal to the system 137 or a computer system 149.

In one embodiment, when a signal is transmitted to the processor 139, it arrives at the multiplexer. The multiplexer is a device that selects between several analog or digital input signals and forwards the selected input to a single output line, making it possible for several input signals to share one device or resource. For example, one analog-to-digital converter or one communications transmission medium is shared across input signals, instead of having one device per input signal. The macrosystem 101 of the present invention may optionally utilize a demultiplexer, which is a device that takes a single input and selects signals of the output of the compatible multiplexer, which is connected to the single input, and a shared selection line. A multiplexer is often used with a complementary demultiplexer on the receiving end. The multiplexer of the present invention makes it possible for the set of noise wires, the set of signal wires and/or the cleaned signal, which can often comprise 80 or more wires, to be paired down to a significantly lower number of wires.

In certain embodiments, an analog multiplexer may be used. An analog multiplexer interprets a continuously variable signal form multiple input sources. A digital multiplexer may also be used. Digital multiplexers interpret signals that usually take only finite levels. For example, an analog signal may be represented by a wave while a digital signal may be represented by a series of ones and zeros.

An amplifier is an electronic device that can increase the magnitude of a signal (a time-varying voltage or current). It is a two-port electronic circuit that uses electric power from a power supply to increase the amplitude (magnitude of the voltage or current) of a signal applied to its input terminals, producing a proportionally greater amplitude signal at its output. The amount of amplification provided by an amplifier is measured by its gain: the ratio of output voltage, current, or power to input. An amplifier is defined as a circuit that has a power gain greater than one. An amplifier can be either a separate piece of equipment or an electrical circuit contained within another device. Amplifiers can be categorized in different ways. One is by the frequency of the electronic signal being amplified.

The amplifier can be more than one type of amplifier. Depending on its placement within the signal chain, the amplifier could be a preamplifier. A preamplifier oftentimes comes first in a signal processing chain and converts a weak signal into a stronger one for further processing.

The amplifier may also be a linear amplifier. A linear amplifier is an electronic circuit whose output is proportional to its input but is capable of delivering more power into a load. Linearity refers to the ability of the amplifier to produce signals that are accurate copies of the input. A linear amplifier responds to different frequency components independently and tends not to generate harmonic distortion or intermodulation distortion. No amplifier can provide perfect linearity however, because the amplifying devices follow nonlinear transfer function and rely on circuitry techniques to reduce those effects.

The amplifier may also be a transimpedance amplifier. A transimpedance amplifier is a current to voltage converter, often implemented with one or more operational amplifiers. A transimpedance amplifier converts the low-level current of a sensor to a voltage however, the voltage is proportional to the input current.

Following amplification of the signal, the signal is transmitted to the analog-to-digital converter (ADC). An ADC is a system that converts an analog signal to a digital signal. Once the signal has been processed via the processor 139, it may be transmitted to the system 137 or to the computer system 149.

The computer system 149 preferably includes at least one computer processor coupled to a memory subsystem containing instructions therein executable by the computer processor to cause the computer system 149 to perform methods of the disclosure. The computer system 149 may optionally any suitable hardware such as the computer processor coupled to random access memory (RAM) and a non-transitory, computer-readable storage system, a graphics card, a sound card, and/or input/output (I/O) devices such as a mouse, a keyboard, speakers, data interconnects, monitors or other displays, and network connections. Preferably, the computer system 149 is operable to display visual images representing patient anatomy and physiology based on the signals and data received from using the catheter.

The visual data may be based in part on data obtained before or during the procedure. The visual data on the computer screen may update dynamically, in real time. Data obtained prior to the procedure may also be used in conjunction with real-time data. Data obtained prior to the procedure may include any type of (two-dimensional or three-dimensional) medical imaging including, but not limited to, computed tomography scans (CT scans), X-rays, magnetic resonance imaging (MRI), ultrasound, elastography, tomography, echocardiograms, and the like. Optionally, visual data obtained during the procedure may be superimposed on previously obtained medical images and displayed on the computer system 149. The computer system 149 may also process other types of data, in addition to visual data, such as electrical data.

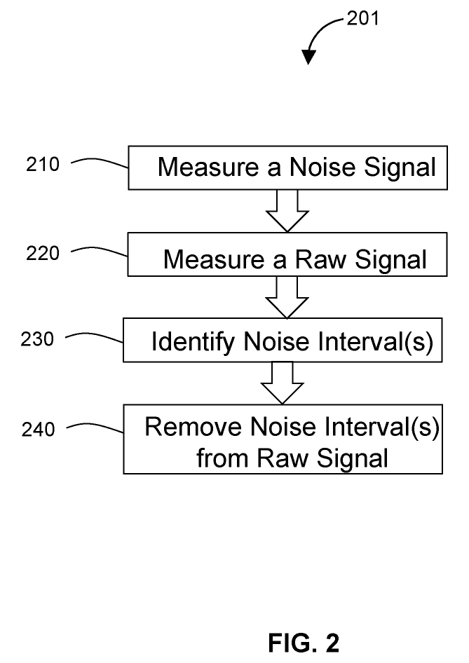

As indicated above, the system 137 is useful to perform methods of the invention. FIG. 2 shows steps of one or more method 201 of the invention. The method 201 includes measuring 210 a noise signal through the set of noise wires and measuring 220 a raw signal through a set of signal wires. The raw signal includes the noise signal. Stated differently, any triboelectric noise carried by the set of noise wires is also carried by the set of signal wires and will be present in the raw signal.

In some embodiments, the noise signal may be filtered to a range of relevant frequencies. For example, the noise signal may be filtered to remove noise created by prevalent power sources or other expected noise frequencies. Specifically, in some embodiments, the noise signal may only be measured at a frequency greater than or equal to 250 Hz, or greater than or equal to 300 Hz.

The method 201 continues by identifying 230 one ore more noise intervals in which the intensity of the noise signal is in excess of a threshold level. The threshold level may be determined by previously processed signals.

While the one or more noise intervals may be identified by any suitable methods, in some embodiments, identifying 230 comprises one or more preprocessing steps selected from: performing a common mode subtraction; applying a high pass filter or low pass filter; squaring the signal; and applying an exponential moving average to the signal. After suitable preprocessing, if any, the predetermined threshold is applied to identify the one or more noise intervals.

The method 201 continues by removing 240 the one or more noise intervals from the raw signal to provide a cleaned signal.

The methods of the invention may include preprocessing the raw signal or further processing the cleaned signal. These additional method steps may include well-known signal manipulation methods including, but not limited to, amplifications, filters (e.g., high/low pass), common mode manipulations (subtraction, rejections, etc.).

A common mode rejection (CMR) operation may improve the spatial resolution of electrogram recordings by decreasing the size of a region of cardiac tissue that contributes to the electrogram recording. The CMR electrode array configuration is able to accurately measure a signal derived exclusively from activation as it passes beneath a central electrode; it eliminates contribution to the local signal by activation of the tissue surrounding a central electrode.

A "central" electrode in the array that detects the local activation signal works in conjunction with multiple "surrounding" contact electrodes that surround the central electrode on the array. It is noted that numerous or any of the various electrodes may each, in turn, be a central electrode for CMR, and while one electrode is being treated as central, the other electrodes that surround it are treated as the surrounding electrodes. As a local signal passes underneath the central electrode, a signal is concurrently recorded from both the central electrode and the surrounding electrodes. The signals from the surrounding electrodes are averaged, and the resulting average is subtracted from the central electrode signal. As a result, the CMR electrode array is able to accurately measure a given signal (e.g., an activation signal when used in cardiac tissue) by eliminating both far-field signal interference and near-field signal interreference from other electrodes on the array. As a result, a signal detected by the central electrode represents only the signal generated by the activation signal as it passes underneath the electrode. The CMR electrode array is thus able to leverage the benefits of unipolar electrodes and bipolar electrodes, while eliminating their drawbacks.

Accordingly, the use of simultaneously obtained electrode data according to the invention is useful to determine relative positions of measurements made by multiple electrodes in the construction of cardiac tissue mapping. A CMR may allow or aid in the computation of conduction velocity. The array can be used to construct a map of cardiac rhythm and tissue properties, such as scarring.

The inventors have recognized that the methods and systems of the invention remove a portion of the raw signal corresponding to periods of relatively high triboelectric noise. Accordingly, if the threshold is higher, fewer periods of triboelectric noise will be identified as noise intervals and more of the raw signal will remain in the cleaned signal. The tradeoff is that the cleaned signal may still include an appreciable period in which the signal also includes triboelectric noise. In contrast, a lower threshold risks removing more of the raw signal than necessary, potentially removing important signal information. Accordingly, an “optimal” threshold level can be established based on previously processed signals from similar environments or from similar equipment.

In operation, the systems and methods of the disclosure may be performed using hardware and/or software elements. Exemplary hardware elements include, but are not limited to, transistors, diodes, vacuum tubes, power sources, resistors, capacitors, sensors/detectors, piezoelectric devices and/or switches. When executed by software elements, the methods may be stored in memory to be executed by a suitable general use processor or may be executable by a specific purpose processor which permanently stores and executes the methods of the invention.

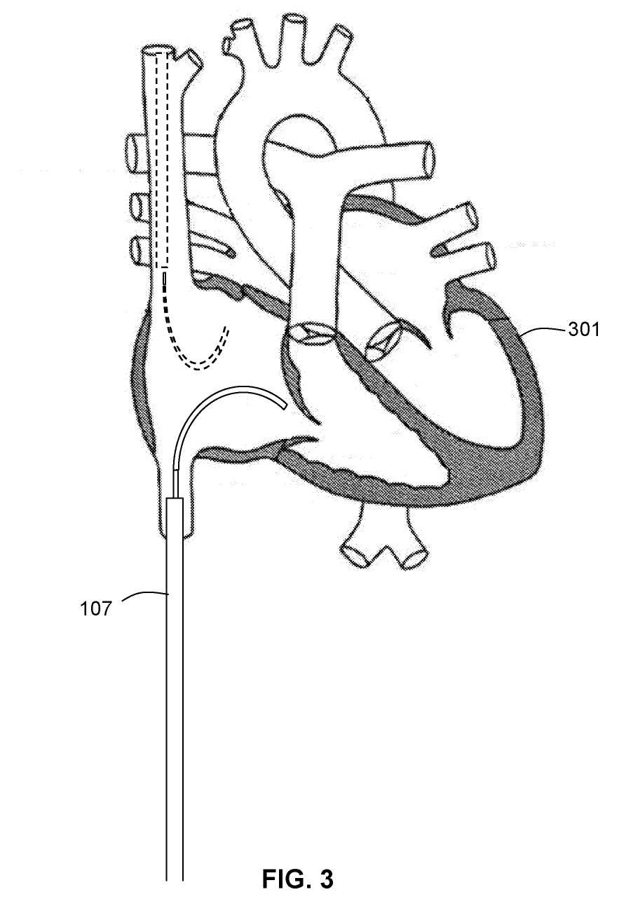

FIG. 3 shows obtaining the measurements of the electrical activity of a heart 301 by placing a catheter 107 of the invention into at least a coronary sinus (CS) of the heart 301 and creating a record of cycle length over a time between about minute and hour. The catheter 107 may be advanced via the inferior vena cava (as drawn solid) or the superior vena cava (drawn in dashed lines). The catheter is operated, optionally held in one position, over a period of time such as a few minutes or about ten minutes or longer. Electrodes on the catheter gather data which may preferably be in the form of one or more electrogram(s) that may be stored and/or viewed on the computer system 149.

What is claimed is:

1. A method of removing triboelectric noise from a signal:
measuring a noise signal through a set of noise wires;
measuring a raw signal through a set of signal wires, the raw signal including the noise signal;
identifying one or more noise intervals over a threshold level in the noise signal; and
removing the one or more noise intervals from the raw signal to provide a cleaned signal.

2. The method of claim 1, wherein the signal is a cardiac activation signal.

3. The method of claim 1, wherein the set of noise wires and the set of signal wires are within a catheter.

4. The method of claim 3, further comprising operating the catheter to create the noise signal.

5. The method of claim 1, wherein the noise signal is measured at a frequency greater than or equal to 300 Hz.

6. The method of claim 1, further comprising analyzing the cleaned signal.

7. The method of claim 1, wherein identifying one or more noise intervals comprises:
performing a common mode subtraction of the noise signal to provide a subtracted signal;
applying a high pass filter to the subtracted signal to provide a filtered signal;
squaring the filtered signal to provide a rectified signal;
applying an exponential moving average to the rectified signal to provide a smoothed signal; and
applying a predetermined threshold to identify one or more noise intervals in the smoothed signal.

8. The method of claim 7, wherein the high pass filter has a cutoff of 300 Hz.

9. The method of claim 7, wherein the predetermined threshold is determined based on previously processed signals.

10. A system for removing triboelectric noise from a signal, the system comprising:
a detector connected to a set of noise wires, the detector configured to identify one or more intervals of noise above a threshold level based on signals from the set of noise wires; and
a subprocessor connected to the detector and a set of signal wires, the subprocessor configured to remove the one or more intervals from signals from the set of signal wires to provide a cleaned signal.

11. The system of claim 10, further comprises a catheter body, the set of signal wires and the set of noise wires running through the catheter body.
